(19) United States
(12) Patent Application Publication
(10) Pub. No.: US 2016/0166518 A1
(43) Pub. Date: Jun. 16, 2016

(54) PROCESS FOR MANUFACTURING PHENYLEPHRINE RESINATE PARTICLES PHENYLEPHRINE RESINATE PARTICLES AND USE OF PHENYLEPHRINE RESINATE PARTICLES IN PHARMACEUTICAL FORMULATIONS

(71) Applicant: JOHNSON & JOHNSON CONSUMER INC., SKILLMAN, NJ (US)

(72) Inventors: Vincent W. Chen, Upper Holland, PA (US); Shun-Por Li, Lansdale, PA (US); Edward A. Koch, Mohnton, PA (US); Der-Yang Lee, Flemington, NJ (US); Joel Waldman, Chalfont, PA (US)

(21) Appl. No.: 14/966,079

(22) Filed: Dec. 11, 2015

Related U.S. Application Data
(60) Provisional application No. 62/091,096, filed on Dec. 12, 2014.

(51) Int. Cl. A61K 31/137; A61K 9/50; A61K 9/14

(52) U.S. Cl. CPC A61K 31/137; A61K 9/146; A61K 9/5047; A61K 9/5042

(57) ABSTRACT

Phenylephrine particles suitable for solid, semi solid or liquid dosage forms are disclosed.

This application claims priority of the benefits of the filing of U.S. Provisional Application Ser. No. 62/091,096, filed Dec. 12, 2014, the entire contents of which are incorporated by reference herein.

### FIELD OF THE INVENTION

The present invention relates to phenylephrine particles suitable for solid, semi solid or liquid dosage forms. The phenylephrine particles, which may be coated, release phenylephrine at rates that provide pharmaceutically suitable plasma concentrations for an extended period of time. The present invention also relates to a process for manufacturing dosage forms containing the phenylephrine particles and to methods for alleviating nasal and respiratory congestion in human subjects with the oral administration of the dosage forms. The dosage forms can further comprise one or more additional therapeutically active agents selected from one or more of the group consisting of antihistamines, decongestants, analgesics, anti-inflammatories, anti-pyretics, cough suppressants and expectorants.

### BACKGROUND OF THE INVENTION

Phenylephrine is a potent vasoconstrictor, possessing both direct and indirect sympathomimetic effects [Hoffman 2001]. The dominant and direct effect is agonism at α1-adrenergic receptors. Stimulation of α1-adrenergic receptors located on capacitance blood vessels of the nasal mucosa (postcapillary venules) results in vasoconstriction, decreased blood volume, and a decrease in the volume of the nasal mucosa (nasal decongestion) [Johnson 1993]. Constricted blood vessels allow less fluid to enter the nose, throat, and sinus linings, which results in decreased inflammation of nasal membranes as well as decreased mucous production [Johnson 1993]. Thus, by constriction of blood vessels, mainly those located in the nasal passages, phenylephrine causes a decrease in nasal congestion [Hoffman 2001, Empey 1981].

Phenylephrine is a Category I (Generally Regarded as Safe and Effective (GRASE)) over-the-counter (OTC) oral nasal decongestant. Globally, phenylephrine has been available since the 1960's, and since 1996, phenylephrine has been widely used in the United States. Phenylephrine hydrochloride, which is widely used in OTC adult and pediatric cough and cold medicines, is indicated for use by adults and children for the temporary relief of nasal congestion due to the common cold, hay fever, or other upper respiratory allergies (allergic rhinitis). It is commercially available in 10 mg tablets for oral administration in adults. The dosing regimen is one 10 mg dose of phenylephrine every four hours, not to exceed 60 mg (six doses) in 24 hours. Complete information is available in the OTC monograph labeling for approved drugs.

Phenylephrine, chemical name (R)-1-(3-hydroxyphenyl)-2-methylaminoethanol, is commercially available as a hydrochloride salt. The empirical formula is C9H13NO2.HCl and the molecular weight is 203.67. The compound, which is a white to off-white crystalline powder, has the following chemical structure:

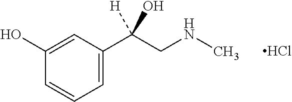

The principal routes of phenylephrine metabolism are sulfate conjugation (mainly in the intestinal wall) and oxidative deamination by both the A and B forms of monoamine oxidase [Suzuki 1979]. Glucuronidation also occurs, but to a lesser extent. In one study, following a 30 mg dose administered orally over eight hours [Ibrahim 1983], phenylephrine was metabolized to phenylephrine-sulfate, m-hydroxymandelic acid, phenylephrine-glucuronide and m-hydroxy-phenylglycol-sulfate at 47%, 30%, 12%, and 6% of the dose, respectively. Deamination is the predominant metabolic pathway after intravenous injection of phenylephrine [Hengstmann 1982], whereas sulfate conjugation is the predominant pathway after oral administration. Phase I and Phase II metabolites of phenylephrine in humans are shown below. The percentage values in the schematic refer to the percent of an oral dose as reported by Ibrahim.

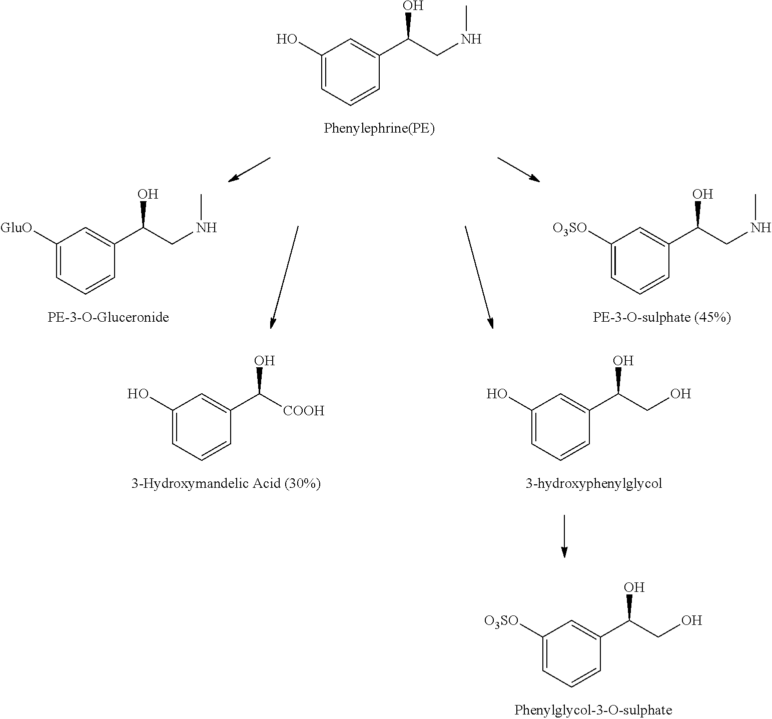

Efficacy data from clinical trials of immediate-release phenylephrine use in adults indicate that phenylephrine is an effective nasal decongestant.

Acetaminophen is a para-aminophenol derivative with analgesic and antipyretic activity. It is used for the temporary relief of minor aches and pains associated with the common cold, backache, headache, toothache, menstrual cramps, and muscular aches; and for the temporary relief of the minor pain of arthritis and for the reduction of fever. The adult dose of acetaminophen in the United States is 1000 mg every four to six hours with a maximum of 4000 mg in 24 hours. The adult dose of extended release acetaminophen is 1300 mg every eight hours with a maximum of 3900 mg in 24 hours.

Acetaminophen is primarily metabolized by the liver via three major parallel pathways: glucuronidation, sulfation, and oxidation [Miners 1983; Slattery 1989; Lee 1992; Miners 1992]. Both the glucuronic and oxidative pathways adhere to a first-order rate process, which means the concentration of acetaminophen metabolized increases as the concentration in the liver increases.

The sulfate pathway adheres to Michaelis-Menten kinetics, which means the concentration of acetaminophen metabolized remains constant once the concentration in the liver increases above a saturation level.

A schematic of acetaminophen metabolism is shown below. Less than 9% of a therapeutic dose is excreted unchanged in the urine [Miners 1992]. The major metabolic pathway is glucuronidation, where 47% to 62% of the acetaminophen dose conjugates with glucuronide. These glucuronide conjugates are inactive and nontoxic [Koch-Weser 1976], and are secreted in bile and eliminated in the urine. Glucuronide conjugation is catalyzed primarily by one isoform of glucuronyltransferase (UGT1A6) [Court 2001] with uridine 5′-diphosphoglucuronic acid as an essential cofactor.

The second major pathway of acetaminophen metabolism is sulfation, where 25% to 36% of the dose conjugates with sulfate. These sulfate ester conjugates are also inactive and nontoxic [Koch-Weser 1976], and are readily excreted in the urine. Sulfation is mediated by sulfotransferases, which are heterogeneous cytosolic enzymes, and 3′-phosphoadenosine 5′-phosphate is a cofactor. Sulfotransferase activity rather than sulfate depletion is the rate-controlling factor of acetaminophen sulfation [Blackledge 1991].

The third pathway is oxidation, where 5% to 8% of the acetaminophen dose is metabolized via the cytochrome P-450 enzyme system. The cytochrome P-450 isoenzyme that is primarily responsible for acetaminophen metabolism is CYP2E1 [Manyike 2000]. When acetaminophen is metabolized by CYP2E1, it forms a highly reactive intermediate, N-acetyl-p-benzoquinoneimine (NAPQI). Because NAPQI is highly reactive, it cannot be measured outside the liver nor can it accumulate. This intermediate is rapidly inactivated by hepatocellular stores of glutathione to form cysteine and mercapturate conjugates, which are both inactive and nontoxic [Koch-Weser 1976]. These conjugates are excreted in the urine [Mitchell 1974].

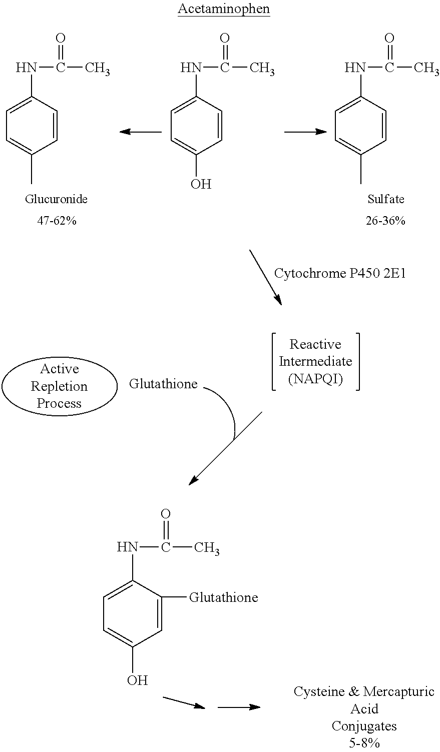

There is a need for less frequent delivery of phenylephrine. Less frequent administration results in improved patient compliance. In addition, constant therapeutic plasma levels of active components can be more effective and even efficacious compared to the fluctuations seen when multiple doses of a conventional immediate release formulation are given. Sustained effective levels could decrease the severity and frequency of side effects seen with high peak plasma levels. Thus, formulations of phenylephrine that can be administered less frequently, for example, once every 6, 8, 12, 16, 20, or 24 hours, are needed.

There is also a need to match the duration of phenylephrine with actives that provide a longer duration than immediate release phenylephrine.

U.S. Published Application No. 20070281020 to Schering-Plough Corporation discloses the administration of a sustained release tablet comprising 30 mg phenylephrine, hydroxypropyl methylcellulose, carboxymethyl cellulose sodium, Kollidon CL-M, colloidal silicon dioxide and magnesium stearate to a human subject and the comparison of the sustained release tablet to three doses of 10 mg immediate release phenylephrine.

U.S. Pat. No. 8,282,957 to McNeil-PPC, Inc. discloses coated phenylephrine particles containing phenylephrine HCl, modified starch and Eudragit NE3OD™ coated with a first coating layer comprising Eudragit RS PO, acetyltributylcitrate and magnesium stearate and a second coating layer comprising Eudragit NE30D™, Eudragit FS30D™, magnesium stearate, sodium lauryl sulfate and simethicone, and use thereof in pharmaceutical dosage forms, including dosage forms containing acetaminophen.

U.S. Pat. No. 6,001,392 to Warner Lambert Company discloses a drug/resin complex that contains a mixture of coated and uncoated Amberlite™ IR69 cross-linked with divinylbenzene.

U.S. Published Application No. 20100068280 to Schering-Plough Corporation discloses pharmaceutical dosage forms comprising phenylephrine in sustained release form. According to an embodiment, a single dose of phenylephrine in a tablet containing 30 mg phenylephrine, lactose monohydrate, Methocel K100M CR, Klucel EXF and magnesium stearate was compared to two 10 mg phenylephrine immediate release tablets dosed 4 hours apart in a bioequivalence study.

U.S. Published Applications Nos. 20050266032 and 20060057205 to Sovereign Pharmaceuticals disclose pharmaceutical dosage forms containing phenylephrine. According to an embodiment, the phenylephrine is incorporated into an ion-exchange resin complex using, e.g., sodium polystyrene sulfonate, and coated with delayed release polymer, e.g., Eudragit® L 100, Kollidon® MAE and Aquacoat® cPD. The formula in this embodiment contains 45 mg sustained release phenylephrine and 15 mg immediate release phenylephrine.

U.S. Pat. No. 8,062,667 to Tris Pharma, Inc. discloses coated drug-ion exchange resin complexes. According to an embodiment, phenylephrine is incorporated into an ion-exchange resin complex using, sodium polystyrene sulfonate, and coated with KOLLICOAT™ SR-30D, triacetin and water.

U.S. Pat. No. 8,394,415 to McNeil-PPC, Inc. discloses a liquid formulation comprising immediate release ibuprofen and an extended release phenylephrine-specified ion exchange resin complex coated with first and second coating layers comprising specified ingredients.

U.S. Published Application No. 20080311201 to McNeil-PPC, Inc. discloses a solid composition comprising ibuprofen (IR) and phenylephrine coated with first coating layer comprising ethylcellulose and second coating layer comprising protective coating.

U.S. Pat. No. 8,883,213 to Coating Place, Inc. discloses a method and composition for loading one or more drugs onto one or more ion exchange resin particles to form a drug loaded resin particle.

U.S. Patent Application No. 20120064167 discloses a controlled release composition comprising phenylephrine and ibuprofen.

U.S. Published Applications Nos. 20140271891; 20140271892; and 20130202700 to McNeil-PPC, Inc. disclose a drug-resin complex that contains phenylephrine and a cation polystyrene sulfonate, wherein the cation polystyrene sulfonate contains particle sizes of about 74 μm to about 177 μm prior to being combined with the phenylephrine. The particles may be coated with a cellulose material such as cellulose acetate and hydroxypropylcellulose.

There continues to be a need for phenylephrine products having the attributes discussed above.

### SUMMARY OF THE INVENTION

The present invention is directed to phenylephrine particles that deliver phenylephrine or a pharmaceutically acceptable salt thereof to a subject in need thereof so as to provide a peak plasma concentration of phenylephrine at about 0.1 to about 16 hours, preferably about 0.5 to about 5 hours, more preferably about 1 to about 4.5 hours, after ingestion and wherein the phenylephrine is maintained at a level greater than about 20, about 40, about 60, about 80, about 100, about 120, about 140, about 160, about 180, or about 200, pg/mL for at least about 6, about 8, about 12, about 16, about 20 and/or about 24 hours after ingestion.

In accordance with a preferred embodiment, the invention is directed to coated phenylephrine resinate particles that deliver phenylephrine or a pharmaceutically acceptable salt thereof to a subject in need thereof so as to provide a peak plasma concentration of phenylephrine at about 0.1 to about 16 hours, preferably about 0.5 to about 5 hours, more preferably about 1 to about 4.5 hours, after ingestion and wherein the phenylephrine is maintained at a level greater than about 20, about 40, about 60, about 80, about 100, about 120, about 140, about 160, about 180, or about 200, pg/mL for at least about 6, about 8, about 12, about 16, about 20 and/or about 24 hours after ingestion.

The present invention is also directed to pharmaceutical dosage forms comprising phenylephrine particles that deliver phenylephrine or a pharmaceutically acceptable salt thereof to a subject in need thereof so as to provide a peak plasma concentration of phenylephrine at about 0.1 to about 16 hours, preferably about 0.5 to about 5 hours, more preferably about 1 to about 4.5 hours, after ingestion and wherein the phenylephrine is maintained at a level greater than about 20, about 40, about 60, about 80, about 100, about 120, about 140, about 160, about 180 or about 200, pg/mL for at least about 6, about 8, about 12, about 16, about 20 and/or about 24 hours after ingestion.

In another embodiment, the phenylephrine particles, which provide extended release of phenylephrine, are combined with phenylephrine in immediate release form.

In another embodiment, the phenylephrine particles are combined with one or more additional therapeutic agent(s) for immediate or sustained release. Such agent or agents may be formulated for immediate release upon ingestion, for sustained release, for release in the colon concomitantly with at least some of the phenylephrine, or any combination thereof. In one embodiment, the additional therapeutic agent is uncoated. In another embodiment, the additional therapeutic agent is coated.

The additional therapeutic agent may be an antihistamine, a decongestant, an analgesic, an anti-inflammatory, an anti-pyretic, a cough suppressant, an expectorant, or any other therapeutic agent or combinations of such agents useful to alleviate the symptoms of a cold, seasonal and other allergies, hay fever, or sinus problems, any of which may cause an increase in nasal discharge. Preferably, the one or more additional therapeutic agents are acetaminophen.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof, and pharmaceutically acceptable salts and esters thereof.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof, and pharmaceutically acceptable salts and prodrugs thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof, and pharmaceutically acceptable salts and prodrugs thereof.

Another aspect of the invention is a method of treating the symptoms of cold, influenza, allergies, or non-allergic rhinitis in a subject in need thereof comprising administering the phenylephrine particles of the invention. In certain embodiments, the phenylephrine particles are administered about every 6, 8, 12, 16, 20, or 24 hours. In one preferred embodiment, the phenylephrine particles are administered about every 12 hours. In another preferred embodiment, the phenylephrine resinate particles are administered about every 8 hours.

Certain embodiments of the invention are methods of maintaining sustained bioavailability of phenylephrine in a subject, comprising orally administering to the subject phenylephrine particles, wherein at least a portion of phenylephrine is absorbed from the colon, and wherein the concentration of phenylephrine in the plasma of the subject is at least about 20, about 40, about 60, about 80, about 100, about 120, about 140, about 160, about 180, or about 200, pg/mL at about 6 hours after administration of the composition. In particular embodiments, the concentration of phenylephrine in the plasma of the subject is at least about 20, about 40, about 60, about 80, about 100, about 120, about 140, about 160, about 180, or about 200, pg/mL at about 8 hours after administration of the composition. In particular embodiments, the concentration of phenylephrine in the plasma of the subject is at least about 20, about 40, about 60, about 80, about 100, about 120, about 140, about 160, about 180, or about 200, pg/mL at about 12 hours after administration of the composition. In particular embodiments, the concentration of phenylephrine in the plasma of the subject is at least about 20, about 40, about 60, about 80, about 100, about 120, about 140, about 160, about 180, or about 200, pg/mL at about 20 hours after administration of the composition. In particular embodiments, the concentration of phenylephrine in the plasma of the subject is at least about 20, about 40, about 60, about 80, about 100, about 120, about 140, about 160, about 180, or about 200, pg/mL at about 24 hours after administration of the composition. Certain other embodiments of the invention are methods of administering phenylephrine to a subject, comprising orally administering phenylephrine particles, said composition delivering at least some of the phenylephrine to the colon where phenylephrine is released in the colon and absorbed from the colon.

The present invention may be more fully understood by reference to the Figures, Detailed Description and Examples which follow.

### BRIEF DESCRIPTION OF THE FIGURES

FIG. 1 shows the effect of particle size distribution of resin on assay content of drug resinate (drug/resin ratio: 1.25:1).

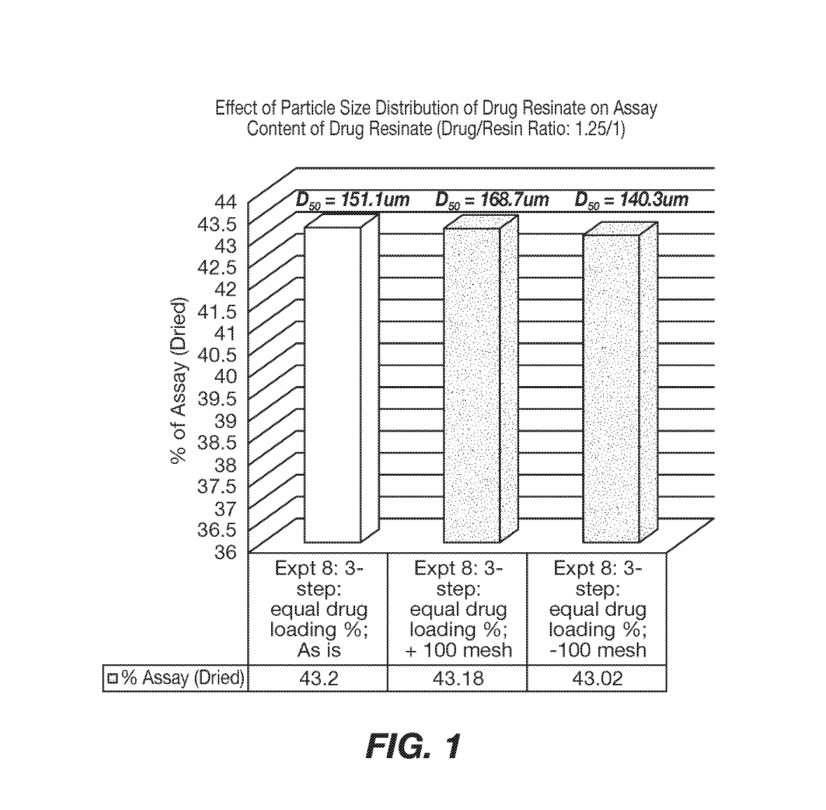

FIG. 2 shows drug loaded v. amount of drug applied at drug:resin ration: 1.33:1 drug/resin ratio (3 step process, pilot scale).

FIG. 3 shows drug loading efficiency in each loading step in a 3-step drug loading process (pilot scale batch).

FIG. 4 shows the effect of drug loading step on % of drug loading efficiency (3-step process at 1.33:1 drug/resin ratio v. 1-step process at four levels of drug/resin ratio).

FIG. 5 shows dissolution profiles for 40% coat level coated phenylephringe resinate (pilot scale) vs. clinical batch (lab scale) utilizing one step loaded resinate.

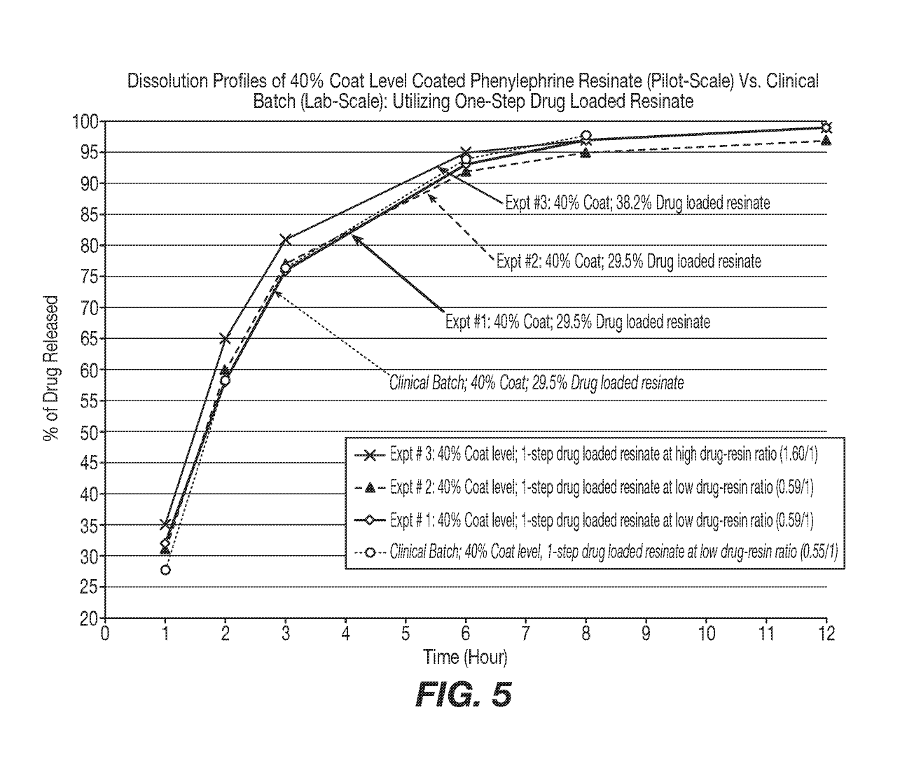

FIG. 6 shows the dissolution profiles of coated phenylephrine resinate at 35, 40, 45 and 50% coat level (pilot scale) utilizing 3-step drug loaded resinate vs. clinical batch 40% coat level (lab scale) utilizing 1-step drug loaded resinate.

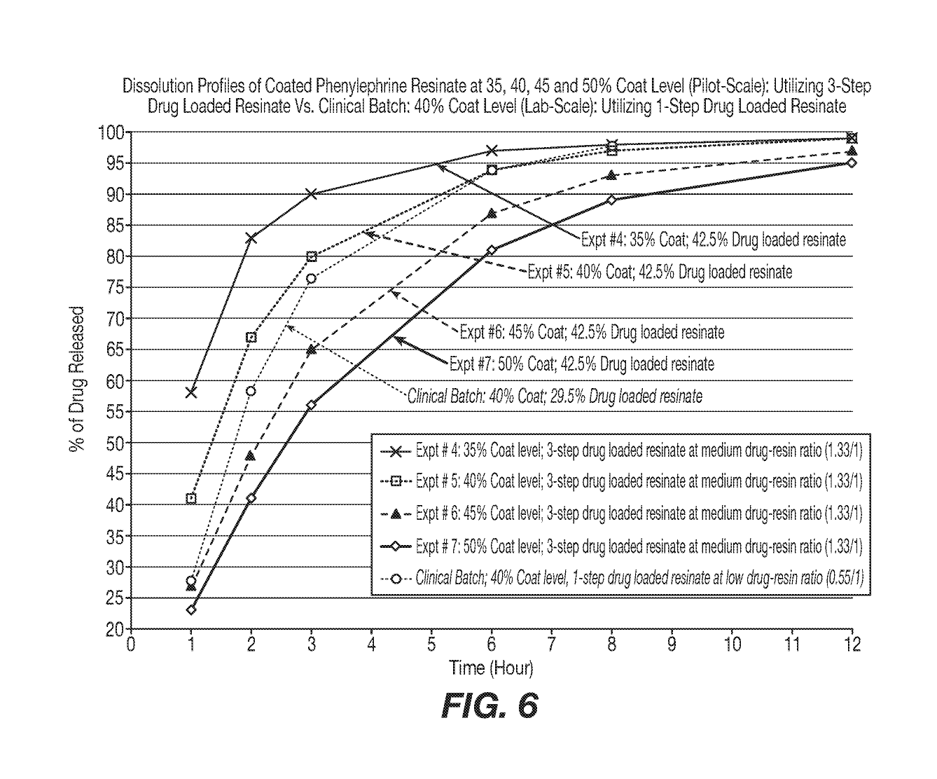

FIG. 7 shows simulated dissolution profiles of tablets containing 40%, 42.5% coated drug resinate utilizing 42.5% drug loaded resinate vs. clinical tablet containing 40% coated drug resinate utilizing 29.5% drug loaded resinate:tablet formula.

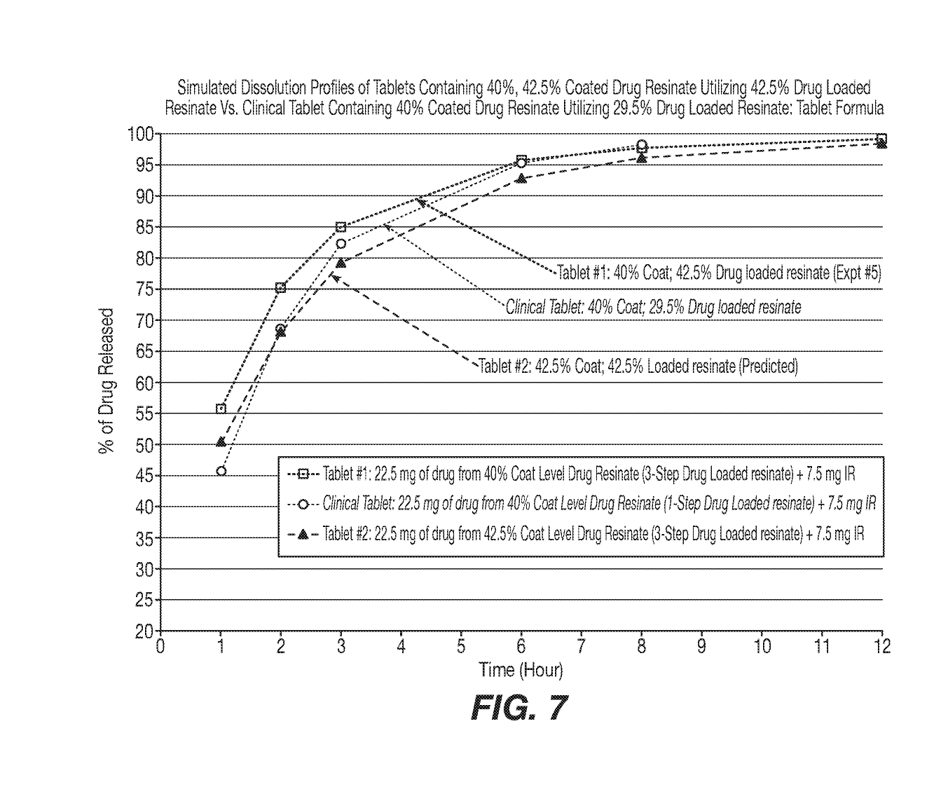

FIG. 8 shows dissolution profiles of coated phenylephrine resinate with various CA/HPC ratios.

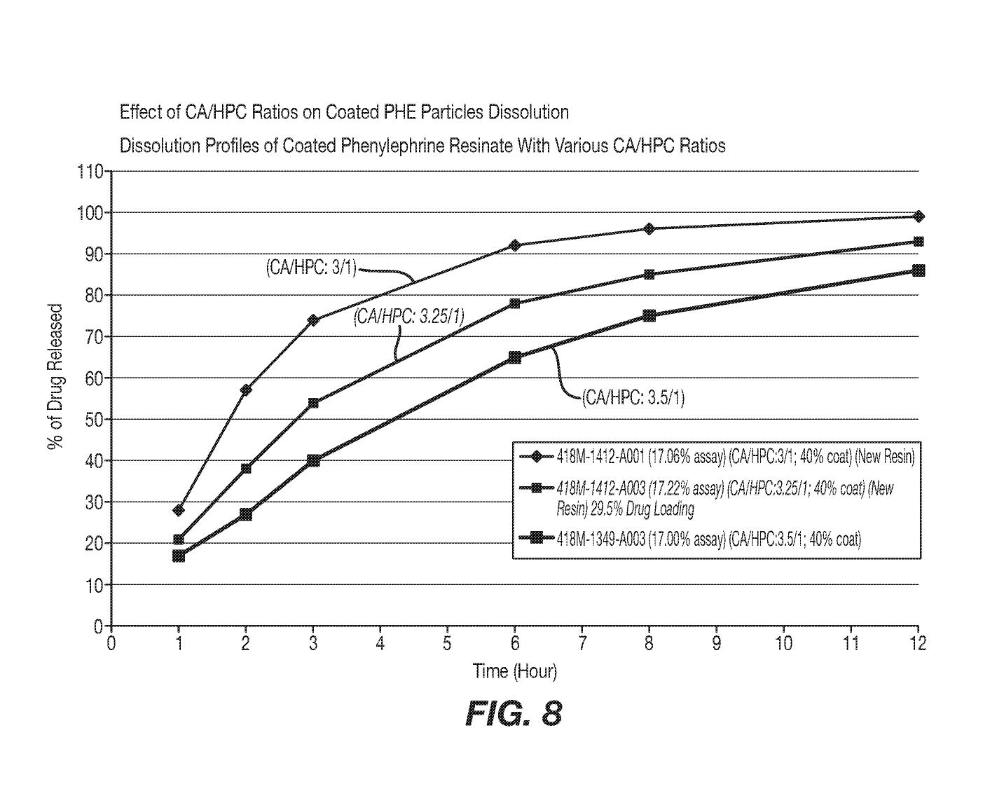

### DETAILED DESCRIPTION OF THE INVENTION

It is believed that one skilled in the art can, based upon the description herein, utilize the present invention to its fullest extent. The following specific embodiments are to be construed as merely illustrative, and not as limiting the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified. In addition, all ranges set forth herein are meant to include any combinations of values between the two endpoints, inclusively.

Definitions

As used herein a pharmaceutically acceptable salt of phenylephrine includes, but is not limited to, phenylephrine hydrochloride, phenylephrine bitartrate, phenylephrine tannate, etc. In one preferred embodiment, the pharmaceutically acceptable salt of phenylephrine is phenylephrine hydrochloride.

“AUC” as used herein means, for any given drug, the “area under the concentration-time curve” from dosing or activation of the drug to a time point, calculated by the trapezoidal rule. AUC is a parameter showing the cumulative plasma concentration of a drug over time, and is an indicator of the total amount and availability of a drug in the plasma.

“Cmax” as used herein means the maximum (or peak) concentration that a drug achieves in tested area after the drug has been administrated and prior to the administration of a second dose.

As used herein, “crystalline form” shall mean the non-amorphous form of the active ingredient such that it displays crystal like properties including, but not limited to, the ability to diffract visible light. Crystalline may also be used to describe an active ingredient in its pure form, i.e., e.g., without the addition of other excipients thereto.

By “delayed release,” it is meant that, after administration, there is at least one period of time when an active ingredient is not being released from the dosage form, i.e., the release of the active ingredient(s) occurs at a time other than immediately following oral administration.

As used herein, “dissolution medium” shall mean any suitable liquid environment in which the suspension dosage form of the present invention can be dissolved, such as, for example, the in vitro dissolution media used for testing of the product, or gastro-intestinal fluids. Suitable in vitro dissolution media used for testing the dissolution of the active ingredient or ingredients from the suspension dosage form of the present invention include those described in the United States Pharmacopeia.

A “dosage”, “dosage form” or “dose” as used herein means the amount of a pharmaceutical composition comprising therapeutically active agent(s) administered at a time. “Dosage”, “dosage form” or “dose” includes administration of one or more units of pharmaceutical composition administered at the same time. In one embodiment, the dosage form is a tablet. In one embodiment the dosage form is a multilayer tablet. In the embodiment comprising a multilayer tablet, one layer may comprise an immediate release portion and another layer may comprise an extended release portion. In the embodiment comprising a multilayer tablet, one layer may comprise the phenylephrine resinate particles, and another layer may comprise an immediate release form of phenylephrine and/or a second active ingredient. In one embodiment the dosage form comprising phenylephrine resinate particles is a liquid filled soft-gel.

As used herein “drug-resin complex” shall mean the bound form of an active ingredient, including but not limited to the pharmaceutical active ingredients, and an ion exchange resin. The drug-resin complex is also referred to in the art as a “resinate.” An ion exchange resin that may be used in accordance with the invention is Amberlite™ IRP 69, The Dow Chemical Company, an insoluble, strongly acidic, sodium form cationic exchange resin derived from sulfonated copolymer of styrene and divinylbenzene. The mobile, or exchangeable cation is sodium, which can be exchanged for, or replaced by, many cationic (basic) species, including, e.g., copper, zinc, iron, calcium, strontium, magnesium and lithium. Adsorption of drug onto ion exchange resin particles to form the drug/resin complex is a well known technique as shown in U.S. Pat. Nos. 2,990,332 and 4,221,778. In general the drug is mixed with an aqueous suspension of the resin, and the complex is then washed and dried. Adsorption of drug onto the resin may be detected by measuring a change in the pH of the reaction medium, or by measuring a change in concentration of sodium or drug. The drug/resin complex formed can be collected and washed with ethanol and/or water to insure removal of any unbound drug. The complexes are usually air-dried in trays at room or elevated temperature. They can also be dried via methods such as centrifugation, filtration, pressurized filtration, oven drying and fluid bed drying. The drug/resin complex has a ratio of phenylephrine to resin of greater than about 1:1, more preferably about 1:1 to about 1.8:1, more preferably about 1.2:1 to about 1.6:1, more preferably about 1.4:1.

“Enteric” shall mean being able to be dissolved at a pH of greater than about 5.0 or greater than about 5.5 or greater than about 6.0 or that which is found in the intestine.

By “extended release,” it is meant that, after administration, an active ingredient is released from the dosage form in a substantially continuous, regulated manner, and the time for complete release, i.e., depletion, of the active ingredient from the dosage form is longer than that associated with an immediate release dosage form of the same. Types of extended release include controlled, sustained, prolonged, zero-order, first-order, pulsatile, and the like.

As used herein, “immediate release” means that the dissolution characteristics of at least one active ingredient meet USP specifications for immediate release tablets containing that active ingredient. An active ingredient having an immediate release property may be dissolved in the gastrointestinal contents, with no intention of delaying or prolonging the dissolution of the active ingredient.

“Liquid dosage forms” may nonexclusively include suspensions or elixirs, wherein one or more of the active ingredients is dissolved, partially dissolved or in an undissolved or suspended state.

As used herein, “modified release” shall apply to the altered release or dissolution of an active ingredient in a dissolution medium, such as gastrointestinal fluids. Types of modified release include: 1) extended release; or 2) delayed release. In general, modified release dosage forms are formulated to make the active ingredient(s) available over an extended period of time after ingestion, which thereby allows for a reduction in dosing frequency compared to the dosing of the same active ingredient(s) in a conventional dosage form. Modified release dosage forms also permit the use of active ingredient combinations wherein the duration of one active ingredient may differ from the duration of another active ingredient.

As used herein, “pharmacodynamics” or “PD” is the study of the relationship between drug concentration at the site of action and the resulting effect.

As used herein, “pharmacokinetics” or “PK” is the study of the time course of drug absorption, distribution, metabolism and excretion.

As used herein, the term “phenylephrine” means benzynemethanol, 3-hydroxy-α-[(methylamino)methyl], and includes, but is not limited to pharmaceutically acceptable salts, esters, isomers or derivatives thereof.

As used herein, a drug “release rate” refers to the quantity of drug released from a dosage form per unit time, e.g., milligrams of drug released per hour (mg/hr). Drug release rates are calculated under in vitro dosage form dissolution testing conditions known in the art. As used herein, a drug release rate obtained at a specified time “following administration” refers to the in vitro drug release rate obtained at the specified time following commencement of an appropriate dissolution test, e.g., those set forth in USP 24 (United States Pharmacopeia 24, United States Pharmacopeia Convention, Inc., Rockville, Md,).

“Semipermeable,” as used herein, shall mean that water can pass through, and other molecules, including salts and the active ingredients described herein, are allowed to slowly diffuse through such a membrane when the membrane is in contact with an appropriate dissolution medium, e.g., gastro-intestinal fluids or in-vitro dissolution media.

“Semi-solid dosage forms” shall mean dosage forms which are highly viscous and share some of the properties of liquids, including but not limited to (1) having the ability to substantially conform to something that applies pressure to it and causes its shape to deform; and (2) lacking the ability to flow as easily as a liquid. Semi-solid dosage forms also share some of the properties of solids, including but not limited to having a higher density and a defined shape. Semi-solids may nonexclusively include gels, chewy dosage forms, pectin based chewy forms, confectionery chewy forms, moldable gelatin type of forms.

“Solid dosage forms” shall mean dosage forms which are substantially solid at room temperature and have a density of at least about 0.5 g/cc. Solid dosage forms may non exclusively include, agglomerated tablets, capsule-like medicaments, powder or granule filled capsules, powder or granule filled sachets, compressed tablets, coated tablets, chewable dosage forms, and fast-dissolving dosage forms.

As used herein, “substantially coated” with regard to particles shall mean that less than about 20%, e.g., less than about 15%, or less than about 1.0% of the surface area of the particle is exposed, e.g., not covered, with a desired coating. As used herein, the term “substantially covers” or “substantially continuous” when used to describe a coating means that the coating is generally continuous and generally covers the entire surface of the core or underlying layer, so that little to none of the active ingredient or underlying layer is exposed. The coatings which are applied to the particles can be layered wherein each layer is prepared in an aqueous (water based) or organic solvent system and added in succession until the desired coating level is achieved.

“Therapeutic effect,” as used herein, shall mean any effect or action of an active ingredient intended to diagnose, treat, cure, mitigate, or prevent disease, or affect the structure or any function of the body.

Specific embodiments of the present invention are illustrated by way of the following examples.

This invention is not confined to the specific limitations set forth in these examples.

### EXAMPLES

Phenylephrine extended release particles were developed in order to formulate into liquid and solid dosage forms. The phenylephrine extended release particles can be used to match duration with other actives (particularly pain actives) which may provide a longer duration than phenylephrine. Such actives include, but are not limited to, acetaminophen, ibuprofen and naproxen and salts and derivatives thereof.

A multiple step loading process was developed in order to (1) increase the phenylephrine loading level; and (2) increase the phenylephrine loading efficiency. Preferably, the process results in a phenylephrine loading efficiency of greater than about 40%, e.g., about 43%.

Dow literature for Amberlite™ IRP69 discloses that use of two or more loading stages, separating the resin from the liquid phase between stages, is an effective means of achieving maximum loading of drug on the resin. See http://www.dow.com/assets/attachments/business/process_chemicals/amberlite_and_duolite_pharmaceutical_grade_resins/amberlite_irp69/tds/amberlite_irp69.pdf. (2006). The present inventors have determined that they can achieve similar drug loading efficiency when using the same amount of drug in multiple loading steps, e.g., when using 1.4 parts drug/1 part resin, and employing a three step loading process; the amount of drug in each step can vary (e.g., 50%, 25% and 25% of the total amount of drug used; or e.g., 33⅓%, 33⅓%, 33¼% of the total amount of drug used) without significantly impacting loading efficiency.

Materials:

(1) Amberlite™ Ion-exchange Resin having particle sizes as set forth in Table A below:

TABLE A
Particle Size Analysis of Sodium Polystyrene Sulfonate USP
Resin using Dry Sieving Method derived from United States
Pharmacopeia <811> and <786>

| Particle Size | | |
|---|---|---|
| Measurement | Target Amount | |
| Determination/Unit (%) | (%) | Result (%) |
| % >0.150 mm | ≦7 | 3 |
| % >0.125 mm | None (report | 24 |
| | value) | |
| % >0.100 mm | None (report | 70 |
| | value) | |
| % <0.075 mm | ≦5 | 2 |

(2) Phenylephrine HCl USP

### Example 1

### Lab Based Production of Loaded Phenylephrine Resinate: 3× Loading at 40° C.

The drug loading steps follow the sequence outlined in Table 1.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container and heated to 40° C.
2. 45.5 g of phenylephrine HCl was added and dissolved while mixing at 40° C. for 10 minutes.
3. The Amberlite™ Ion-exchange Resin was added while slowly mixing, and the mixer speed was adjusted to maintain a vigorous flow.
4. After 60 minutes of mixing, the contents were filtered under vacuum.

Step B
1. 200.0 g of purified water was added to a suitable sized container and heated to 40° C.
2. 31.5 g of phenylephrine HCl was added and dissolved while mixing at 40° C. for 10 minutes.
3. The wet loaded resinate from Step A was added while slowly mixing, and the mixer speed was adjusted to maintain a vigorous flow.
4. After 60 minutes of mixing, the contents were filtered under vacuum.

Step C

1. 200.0 g of purified water was added to a suitable sized container and heated to 40° C.

2. 10.5g of phenylephrine HCl was added and dissolved while mixing at 40° C. for 10 minutes.

3. The wet loaded resinate from Step B was added while slowly mixing, and the mixer speed was adjusted to maintain a vigorous flow.

4. After 60 minutes of mixing, the contents were filtered under vacuum.

5. The filtered contents were washed 5 times with 200 mL portions of purified water. The washed drug loaded resin was collected and allowed to oven dry at 40° C. for 24 hours.

TABLE 1
Formula Loading Steps; 1.25:1 Drug:resin ratio
(87.5 g phenylephrine and 70 g of raw resinate)

| | | | Water | Phenyl- | % (w/w) |
|---|---|---|---|---|---|
| | % Phenyl- | Phenylephrine | Amount | ephrine + | Drug |
| Step | ephrine | Amount (g) | (g) | Water (g) | Solution |
| Part B: | 52 | 45.5 | 200 | 245.5 | 18.5 |
| Step A | | | | | |
| Part B: | 36 | 31.5 | 200 | 231.5 | 13.6 |
| Step B | | | | | |
| Part B: | 12 | 10.5 | 200 | 210.5 | 50 |
| Step C | | | | | |
| Total | 100 | 87.5 | | | |
| Part B | NA | 12.25* | 200 | 210.5 | 6.125 |

*A drug/resin ratio at 1.6/1 represents 112 g of drug and 70 g of raw resinate. An additional 24.5 g of drug is needed to achieve 1.6/1 loading for the Part A resinate since the half of the drug loaded resinate was removed for analytical test. Therefore, only half the amount of 24.5 g of drug (12.5 g) was needed to complete the drug loading process to achieve 1.6/1 loading.

### Example 2

### Lab Based Production of Loaded Phenylephrine Resinate: 3× Loading at Room Temperature (25° C.)

The drug loading steps follow sequence outlined in Table 1.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container at room temperature.
2. 45.5 g of Phenylephrine HCl was added and dissolved while mixing at room temperature for 10 minutes.
3. The Amberlite™ Ion-exchange Resin was added while slowly mixing, and the mixer speed was adjusted to maintain a vigorous flow.
4. After 60 minutes of mixing, the contents were filtered under vacuum.

Step B
1. 200g of purified water was added to a suitable sized container at room temperature.
2. 31.5 g of Phenylephrine HCl was added and dissolved while mixing for 10 minutes.
3. The wet loaded resinate from Step A was added while slowly mixing, and the mixer speed was adjusted to maintain a vigorous flow.
4. After 60 minutes of mixing, the contents were filtered under vacuum.

Step C
1. 200 g of purified water was added to a suitable sized container at room temperature.
2. 10.5 g of Phenylephrine HCl was added and dissolved while mixing for 10 minutes.
3. The wet loaded resinate from Step B was added while slowly mixing, and the mixer speed was adjusted to maintain a vigorous flow.
4. After 60 minutes of mixing, the contents were filtered under vacuum.
5. The filtered contents were washed 5 times with 200 mL portions of purified water.

The washed drug loaded resin was collected and allowed to oven dry at 40° C. for 24 hours.

### Example 3

### Lab Based Production of Loaded Phenylephrine Resinate: 3× Loading at Room Temperature (25° C.) with lower mixing times

The steps for Example 2 were followed for Example 3, and the 60 minute mixing time for each step was reduced from 60 minutes to 15 minutes.

### Example 4

### Lab Based Production of Loaded Phenylephrine Resinate: 3× Loading at Room Temperature (25° C.) with Reduction in Filtration Steps

The steps for Example 2 were followed for Example 4, and the filtration steps in Step A and Step B were eliminated.

### Example 5

### Lab Based Production of Loaded Phenylephrine Resinate: 3× Loading at Room Temperature (25° C.) with Equal Amounts of Phenylephrine in each Loading Step

The drug loading steps follow the sequence outlined in Table 2.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container at room temperature.
2. 29.2 g of Phenylephrine HCl was added and dissolved while mixing at room temperature for 10 minutes.
3. The Amberlite™ Ion-exchange Resin was added while slowly mixing, and the mixer speed was adjusted to maintain a vigorous flow.
4. After 60 minutes of mixing, the contents were collected.

Step B
1. 200g of purified water was added to a suitable sized container at room temperature.
2. 29.2 g of Phenylephrine HCl was added and dissolved while mixing for 10 minutes.
3. The wet loaded resinate from Step A was added while slowly mixing, and the mixer speed was adjusted to maintain a vigorous flow.
4. After 60 minutes of mixing, the contents were collected.

Step C
1. 200 g of purified water was added to a suitable sized container at room temperature.
2. 29.1 g of Phenylephrine HCl was added and dissolved while mixing for 10 minutes.
3. The wet loaded resinate from Step B was added while slowly mixing, and the mixer speed was adjusted to maintain a vigorous flow.
4. After 60 minutes of mixing, the contents were filtered under vacuum.
5. The filtered contents were washed 5 times with 200 mL portions of purified water. The washed drug loaded resin was collected and allowed to oven dry at 40° C. for 24 hours.

TABLE 2
Formula Loading Steps; 1.25:1 Drug:resin ratio
(87.5 g phenylephrine and 70 g of raw resinate)

| | | Phenyl- | | | |
|---|---|---|---|---|---|
| | | ephrine | Water | Phenyl- | % (w/w) |
| | % Phenyl- | Amount | Amount | ephrine + | Drug |
| Step | ephrine | (g) | (g) | Water (g) | Solution |
| Part B: Step A | 33.37 | 29.2 | 200 | 229.2 | 12.7 |
| Part B: Step B | 33.37 | 29.1 | 200 | 229.2 | 12.7 |
| Part B: Step C | 33.26 | 29.2 | 200 | 229.1 | 12.7 |
| Total | 100 | 87.5 | | | |

* Drug/resin ratio: Step A: 29.2/70 = 0.417/1; Step B: 58.4/70 = 0.834/1; Step C: 87.5/70 = 1.25/1.

### Example 6

### Lab Based Production of Loaded Phenylephrine Resinate: 2× Loading at Room Temperature (25° C.)

The drug loading steps follow sequence outlined in Table 3.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container and heated to 40° C.
2. 45.5 g of Phenylephrine HCl was added and dissolved while mixing at room temperature for 10 minutes.
3. The Amberlite™ Ion-exchange Resin was added while slowly mixing, and the mixer speed was adjusted to maintain a vigorous flow.
4. After 60 minutes of mixing, the contents were collected.

Step B
1. 200 g of purified water was added to a suitable sized container at room temperature.
2. 42.0 g of Phenylephrine HCl was added and dissolved while mixing for 10 minutes.
3. The wet loaded resinate from Step A was added while slowly mixing, and the mixer speed was adjusted to maintain a vigorous flow.
4. After 60 minutes of mixing, the contents were filtered under vacuum.
5. The filtered contents were washed 5 times with 200 mL portions of purified water.

The washed drug loaded resin was collected and allowed to oven dry at 40° C. for 24 hours.

TABLE 3
Formula Loading Steps; 1.25:1 Drug:Resin ratio
(87.5 g phenylephrine and 70 g of raw resinate)

| | | Phenyl- | | | |
|---|---|---|---|---|---|
| | | ephrine | Water | Phenyl- | % (w/w) |
| | % Phenyl- | Amount | Amount | ephrine + | Drug |
| Step | ephrine | (g) | (g) | Water(g) | Solution |
| Part B: Step A | 52 | 45.5 | 200 | 245.5 | 18.5 |
| Part B: Step B | 48 | 42.0 | 200 | 242 | 17.4 |
| Total | 100 | 87.5 | | | |

*Drug resin ratio: 45.5/70 = 0.65; 42/70 = 0.6; 0.65 + 0.6 = 1.25

### Example 7

### Lab Based Production of Loaded Phenylephrine Resinate: 1× Loading at Room Temperature (25° C.)

The drug loading steps follow sequence outlined in Table 4.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container and heated to 40° C.
2. 87.5 g of Phenylephrine HCl was added and dissolved while mixing at Room temperature for 10 minutes.
3. The Amberlite™ Ion-exchange Resin was added while slowly mixing, and the mixer speed was adjusted to maintain a vigorous flow.
4. After 60 minutes of mixing, the contents were filtered under vacuum. 5. The filtered contents were washed 5 times with 200 mL portions of purified water. The washed drug loaded resin was collected and allowed to oven dry at 40° C. for 24 hours.

TABLE 4
Formula Loading Steps; 1.25:1 Drug:Resin ratio
(87.5 g phenylephrine and 70 g of raw resinate)

| | | Phenyl- | | Phenyl- | |
|---|---|---|---|---|---|
| | | ephrine | Water | ephrine + | % (w/w) |
| | % Phenyl- | Amount | Amount | Water | Drug |
| Step | ephrine | (g) | (g) | (g) | Solution |
| Part B: Step A | 100 | 87.5 | 200 | 287.5 | 30.4 |
| Total | 100 | 87.5 | | | |

*Drug resinate ratio: 45.5/70 = 0.65; 42/70 = 0.6; 0.65 + 0.6 = 1.25

### Example 8

### Lab Based Production of Loaded Phenylephrine Resinate: 4× Loading at Room Temperature (25° C.)

The drug loading steps follow sequence outlined in Table 5.

Part A: Washing of Resinate
1. 200.0 g of purified water was weighed in a suitable sized container.
2. While mixing, 70.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 200.0 g of purified water was added to a suitable sized container and heated to 40° C.
2. 21.9 g of Phenylephrine HCl was added and dissolved while mixing at room temperature for 10 minutes.
3. The Amberlite™ Ion-exchange Resin was added while slowly mixing, and the mixer speed was adjusted to maintain a vigorous flow.
4. After 60 minutes of mixing, the contents were collected.

Step B
1. 200g of purified water was added to a suitable sized container at room temperature.
2. 21.9 g of Phenylephrine HCl was added and dissolved while mixing for 10 minutes.
3. The wet loaded resinate from Step A was added while slowly mixing, and the mixer speed was adjusted to maintain a vigorous flow.
4. After 60 minutes of mixing, the contents were collected.

Step C
1. 200 g of purified water was added to a suitable sized container at Room temperature.
2. 21.9 g of Phenylephrine HCl was added and dissolved while mixing for 10 minutes.
3. The wet loaded resinate from Step B was added while slowly mixing, and the mixer speed was adjusted to maintain a vigorous flow.
4. After 60 minutes of mixing, the contents were collected.

Step D
1. 200 g of purified water was added to a suitable sized container at Room temperature.
2. 21.8 g of Phenylephrine HCl was added and dissolved while mixing for 10 minutes.
3. The wet loaded resinate from Step C was added while slowly mixing, and the mixer speed was adjusted to maintain a vigorous flow.
4. After 60 minutes of mixing, the contents were filtered under vacuum.
5. The filtered contents were washed 5 times with 200 mL portions of purified water. The washed drug loaded resin was collected and allowed to oven dry at 40° C. for 24 hours.

TABLE 5
Formula Loading Steps; 1.25:1 Drug:resin ratio
(87.5 g phenylephrine and 70 g of raw resinate)

| | | Phenyl- | | Phenyl- | |
|---|---|---|---|---|---|
| | | ephrine | Water | ephrine + | % (w/w) |
| | % Phenyl- | Amount | Amount | Water | Drug |
| Step | ephrine | (g) | (g) | (g) | Solution |
| Part B: Step A | 25 | 21.9 | 200 | 221.9 | 9.9 |
| Part B: Step B | 25 | 21.9 | 200 | 221.9 | 9.9 |
| Part B: Step C | 25 | 21.9 | 200 | 221.9 | 9.9 |
| Part B: Step D | 25 | 21.9 | 200 | 221.9 | 9.9 |
| Total | 100 | 87.5 | | | |

*Drug/resin ratio: Step A: 21.9/70 = 0.312/1; Step B: 43.8/70 = 0.625/1; Step C: 65.7/70 = 0.938/1; Step D: 87.5/1

### Example 9

### Lab Based Production of Loaded Phenylephrine Resinate: 3× Loading in Equal Amounts of Phenylephrine, with Reduced Mixing Times

The drug loading steps follow sequence outlined in Table 6.

Part A: Washing of Resinate
1. 114.0 g of purified water was weighed in a suitable sized container.
2. While mixing, 40.0 g of Amberlite™ Ion-exchange Resin was slowly added and mixed for 15 minutes.
3. The contents were transferred to a filtering funnel and filtered under vacuum to form a wet cake.
4. The wet cake was rinsed with 200.0 g of purified water (Wash 1).
5. The wet cake was again rinsed with 200.0 g of purified water (Wash 2).

Part B: Drug Loading on Resin

Step A
1. 114.0 g of purified water was added to a suitable sized container at room temperature.
2. 16.67 g of Phenylephrine HCl was added and dissolved while mixing at room temperature for 10 minutes.
3. The Amberlite™ Ion-exchange Resin was added while slowly mixing, and the mixer speed was adjusted to maintain a vigorous flow.
4. After 30 minutes of mixing, the contents were filtered under vacuum.

Step B
1. 114.0 g of purified water was added to a suitable sized container at room temperature.
2. 16.67 g of Phenylephrine HCl was added and dissolved while mixing at room temperature for 10 minutes.
3. The wet loaded resinate from Step A was added while slowly mixing, and the mixer speed was adjusted to maintain a vigorous flow.
4. After 30 minutes of mixing, the contents were filtered under vacuum.

Step C
1. 114.0 g of purified water was added to a suitable sized container at room temperature.
2. 16.66 g of Phenylephrine HCl was added and dissolved while mixing at room temperature for 10 minutes.
3. The wet loaded resinate from Step B was added while slowly mixing, and the mixer speed was adjusted to maintain a vigorous flow.
4. After 30 minutes of mixing, the contents were filtered under vacuum.
5. The filtered contents were washed 5 times with 200 mL portions of purified water. The washed drug loaded resin was collected and allowed to oven dry at 40° C. for 24 hours.

TABLE 6
Formula Loading Steps; 1.25:1 Drug:resin ratio
(50.0 g phenylephrine and 40 g of raw resinate)

| | | Phenyl- | | Phenyl- | |
|---|---|---|---|---|---|
| | | ephrine | Water | ephrine + | % (w/w) |
| | % Phenyl- | Amount | Amount | Water | Drug |
| Step | ephrine | (g) | (g) | (g) | Solution |
| Part B: Step A | 33.37 | 16.67 | 114 | 130.27 | 12.7 |
| Part B: Step B | 33.37 | 16.67 | 114 | 130.27 | 12.7 |
| Part B: Step C | 33.26 | 16.66 | 114 | 130.26 | 12.7 |
| Total | 100 | 50.0 | | | |

*Drug/resin ratio: Step A: 16.67/40 = 0.417/1; Step B: 33.34/40 = 0.834/1; Step C: 50/40 = 1.25/1

### Example 10

### Lab Based Production of Loaded Phenylephrine Resinate: 3× Loading in Equal Amounts of Phenylephrine, with Reduced Mixing Times

The drug loading steps follow sequence outlined in Table 6, with an additional lot of resin.

### Example 11

### Lab Based Production of Loaded Phenylephrine Resinate: 3× Loading in Equal Amounts of Phenylephrine, with Reduced Mixing Times

The drug loading steps follow sequence outlined in Table 6, with an additional lot of resin.

TABLE 7
Assay results for Phenylephrine:
The examples above were tested for % phenylephrine
to determine the amount loaded onto the resinate
as a function of steps:

| | | % Phenyl- |
|---|---|---|
| | | ephrine |
| Example | Description | Assay |
| Example 1 | 3 Step Loading at 40° C. | 42.70 |
| Example 2 | 3 Step Loading at room temperature | 42.96 |
| Example 3 | 3 Step Loading at lower mixing times | 42.28 |
| Example 4 | 3 Step Loading with reduced filtration | 39.99 |
| | steps | |
| Example 5 | 3 Step Loading with equal drug loading | 43.01 |
| | amounts in each step | |
| Example 6 | 2 Step Loading at room temperature | 41.76 |
| Example 7 | 1 Step Loading at room temperature | 37.42 |
| Example 8 | 4 Step Loading at room temperature | 43.87 |
| Example 9 | 3 Step Loading with equal amounts and | 43.2 |
| | reduced mixing times | |
| Example 9 (a) | Example 9 after sieving above 100 mesh | 43.18 |
| Example 9 (b) | Example 9 after sieving below 100 mesh | 43.02 |
| Example 10 | 3 Step Loading with equal drug loading | 42.70 |
| | amounts in each step, new resin lot | |
| Example 11 | 3 Step Loading with equal drug loading | 42.98 |
| | amounts in each step, new resin lot | |

### Example 12 (A and B)

### Production Scale of Loaded Phenylephrine Resinate: 3× Loading in Equal Amounts of Phenylephrine, with Reduced Mixing Times

The drug loading steps follow sequence outlined in Table 8.

12A

Part A: Washing of Resinate
1. 36.0 kg of purified water was weighed in a 50 gallon kettle equipped with a pneumatic mixer.
2. While mixing, 18.0 kg of Amberlite™ Ion-exchange Resin (Anhydrous) resin was slowly added and mixed for 30 minutes.
3. The contents were transferred into a filtering chamber and filtered to form a wet cake.
4. The wet cake was rinsed with 4.0 kg of purified water (Wash 1), and filtered using compressed air.
5. The wet cake was again rinsed with 36.10 kg of purified water (Wash 2)

Part B: Drug Loading on Resin

Step A
1. 69.1 kg of purified water was added to a 55 gallon stainless steel tank equipped with a pneumatic mixer.
2. 123.94 kg of phenylephrine HCl was added and dissolved while mixing at room temperature for 10 minutes to form the phenylephrine solution.
3. 31.0 kg of Phenylephrine HCl solution from Step 2 was added to a 50 gallon kettle
4. The Amberlite™ Ion-exchange Resin was added while slowly mixing.
5. After 30 minutes of mixing, the contents were transferred to a filtration chamber and filtered using compressed air.

Step B
1. 31.0 kg of the phenylephrine solution from Step A (2) was added to a 50 gallon kettle at room temperature.
2. 35.45 kg of wet loaded resinate from Step A was added while slowly mixing.
3. After 30 minutes of mixing, the contents were transferred to a filtration chamber and filtered using compressed air.

Step C
1. 30.7 kg of the phenylephrine solution from Step A (2) was added to a 50 gallon kettle at room temperature.
2. 39.17 kg of wet loaded resinate from Step B was added while slowly mixing, and the mixer speed was adjusted to maintain a vigorous flow.
3. After 30 minutes of mixing, the contents were transferred to a filtration chamber and filtered using compressed air.
4. Washing #1: 22.0 kg of purified water was added to a filtration chamber containing the wet resinate from Step 3 and filtered using compressed air.
5. Washing #2: 22.0 kg of purified water was added to a filtration chamber containing the wet resinate from Step 4 and filtered using compressed air.
6. Washing #3: 22.0 kg of purified water was added to a filtration chamber containing the wet resinate from Step 5 and filtered using compressed air.
7. Washing #4: 22.0 kg of purified water was added to a filtration chamber containing the wet resinate from Step 6 and filtered using compressed air.
8. The wet resinate was transferred into a fluid bed dryer for drying at an inlet temperature of 140° F., a fluidizing air volume of 550 cfm and an end point of external air temperature of 110° F.

TABLE 8
Formula Loading Steps; 1.33:1 Drug:resin ratio
(23.94 kg phenylephrine and 18 kg of raw resinate anhydrous)

| | | Phenyl- | | Phenyl- | |
|---|---|---|---|---|---|
| | % | ephrine | Water | ephrine + | % (w/w) |
| | Phenyl- | Amount | Amount | Water | Drug |
| Step | ephrine | (kg) | (kg) | (kg) | Solution |
| Part B: Step A | 33.37 | 7.98 | 23.03 | 31.01 | 25.7 |
| Part B: Step B | 33.37 | 7.98 | 23.03 | 31.01 | 25.7 |
| Part B: Step C | 33.26 | 7.98 | 23.03 | 30.70 | 25.7 |
| Total | 100 | 23.94 | | | |

*Drug/resin ratio: Step A: 7.98/18 = 0.443/1; Step B: 15.96/18 = 0.887/1; Step C: 23.94/18 = 1.33/1

TABLE 9
Assay results for Phenylephrine: Samples pulled and
analyzed between each loading step

| | | % Phenyl- |
|---|---|---|
| | Sample for | ephrine |
| Example | analysis | Assay |
| Example 12: 3 Step Equal Drug Loading | First Step | 24.60 |
| Example 12: 3 Step Equal Drug Loading | Second Step | 37.13 |
| Example 12: 3 Step Equal Drug Loading | Third Step | 42.19 |

12B

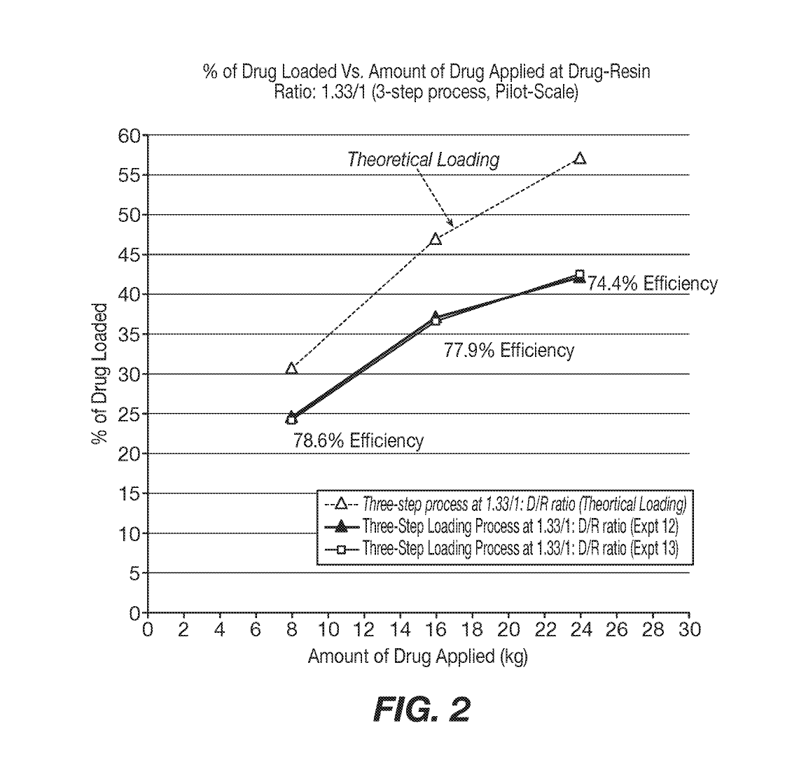

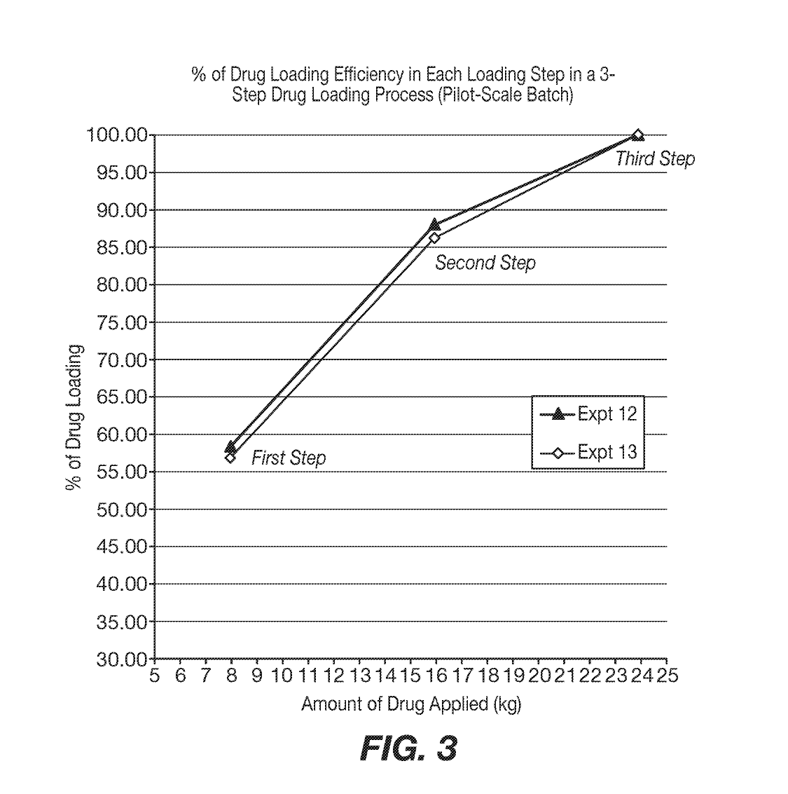

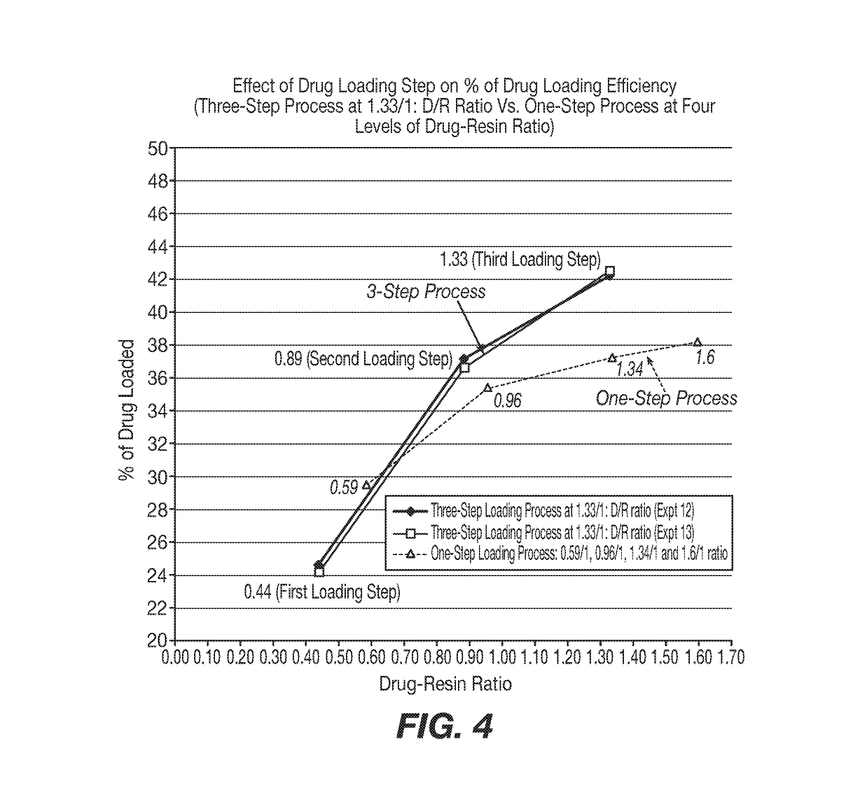

Example 12A was repeated to obtain the data represented in FIGS. 2-4.

Discussion

The results above demonstrate that:

(1) a multiple step loading process increases the level of phenylephrine in the particles, i.e., 4-step>3-step>2-steps>single step when a fixed drug/resin ratio is applied;

(2) a rinse between the loading increases the level of phenylephrine in the particles due to the removal of counter ions;

(3) the drug/resin ratio is a factor determining the loading level while the temperature and mixing time have no significant impact;

(4) with 4 different lots of resin, no significant difference on the phenylephrine loading level was observed within the range of sodium content used in the study;

(5) similar results are achieved between lab-scale and pilot-scale, i.e., the process can be scaled up 450× with minimum modifications;

(6) resin particle size difference in this study does not affect the loading efficiency;

(7) the first step of loading has higher efficiency, with each additional step, the increase on the loading efficiency decreases. This may be contributed from the availability and accessibility of the binding sites in the resin;

(8) a single step loading process seems to have limitations on the phenylephrine loading level at higher drug/resin ratio, while a multiple step process achieves higher loading level with same drug/resin ratio.

Conclusion

With varied drug/resin ratios, targeted drug loading levels of phenylephrine HCl can be obtained with higher efficiency via a multiple-step loading process. Multiple-step loading can reduce the cost and usage of the ion-exchange resin in the formulation and achieve the loading level required to meet the published regulatory limit for polistirex resin in a dosage form.

### Example 13

### Coating of Phenylephrine Resinate Particles

The impact of higher loading of phenylephrine via the multiple step loading process on in-vitro drug release profiles using a cellulose acetate/hydroxypropyl cellulose co-polymeric system (CA/HPC) was observed.

Experiments were performed using the same coating formula (i.e., CA/HPC: 3/1 in a 90/10 acetone/water system) and similar process equipment and parameters on two single step loaded resinates (phenylephrine levels of 29% and 38% w/w, respectively) and one multiple-step loaded resinate (phenylephrine level of 43% w/w)

The formulation performance was evaluated by the in-vitro release profile of phenylephrine up to 24 hours.

Part A: Preparation of Coating Solution

A coating solution containing cellulose actetate and hydroxypropylcellulose in a ratio of 3:1 was prepared as follows.
1. Purified water and acetone were added to a stainless steel container.
2. Hydroxypropylcellulose NF was slowly added to the container and mixed until dissolved.
3. Cellulose Acetate NF was slowly added and mixed until dissolved.
4. Acetone was added until the solution was at the desired weight.
5. The final solution concentration was 6% solids in solution (4.5% cellulose acetate and 1.5% hydroxypropylcellulose).

Part B: Coating of Resinate Particles

Phenylephrine resinate particles prepared according to Tables 10 and 11 were coated using a fluid bed 18 inch Wurster coating unit. The following process parameters were followed during coating:
Inlet Air temperature: 38° C.
Spray rate of solution: 220 g/minute
Outlet air temp: 28° C.
Atomization air pressure: 80 psi
Initial coating change weight: 19.0 kg
Dewpoint: 32° C. (0° C.) is desired
Drying conditions to less than 500 ppm acetone (e.g., 24-48 hours at 60° C. in an oven)
Screening to remove agglomerations

TABLE 1
Formulation Information for Pilot-Scale Coating
Operations Utilizing Drug Loaded Resin from
One-Step Loading Process

| | Coated Drug | | Drug | | |
|---|---|---|---|---|---|
| | Loaded Resinate | | Loaded Resinate | | |
| | CA/HPC | | Drug | | Drug- |
| | Coating | Assay | Loading | Assay | Resin |
| Expt # | Level (%) | (% w/w) | Process | (% w/w) | Ratio |
| 1 | 40 | 16.66 | One-Step | 29.22 | 0.59/1 |
| 2 | 40 | 16.93 | One-Step | 29.46 | 0.59/1 |
| 3 | 40 | 21.95 | One-Step | 38.19 | 1.60/1 |

TABLE 2
Formulation Information for Pilot-Scale Coating
Operations Utilizing Drug Loaded Resin from
Three-Step Loading Process

| | Coated Drug | | Drug | | |
|---|---|---|---|---|---|
| | Loaded Resinate | | Loaded Resinate | | |
| | CA/HPC | | Drug | | Drug- |
| | Coating | Assay | Loading | Assay | Resin |
| Expt # | Level (%) | (% w/w) | Process | (% w/w) | Ratio |
| 4* | 35 | 27.7 | Three-Step | 42.49 | 1.33/1 |
| 5 | 40 | 24.7 | Three-Step | 42.43 | 1.33/1 |
| 6 | 45 | 22.1 | Three-Step | 42.19 | 1.33/1 |
| 7 | 50 | 19.9 | Three-Step | 42.49 | 1.33/1 |

*In-Process Sample

Tables 10 and 11 are set forth below.

The quantitative formula and batch formula are represented in Table 12 and Table 13, respectively.

TABLE 12
Coated Phenylephrine Resinate Quantitative Formula

| | Formula¹ | Weight % |
|---|---|---|
| Component | Mg/Unit | (w/w) |
| Phenylephrine HCL USP | 22.5 | 24.7 |
| Sodium Polystyrene Sulfonate USP | 26.40 | 35.3 |
| (Amberlite ™ Ion-exchange Resin) | | |
| Cellulose Acetate NF | 22.43 | 30 |
| Hydroxypropyl Cellulose NF | 7.48 | 10 |
| Acetone NF³ | — | — |
| Purified Water USP³ | — | — |

¹Unit doses of particles containing 22.5 mg phenylephrine HCL is approximately 74.48 mg. Actual weight is dependent on the assayed amount of phenylephrine HCL in the particles.
²Quantity represents the free base (1 mg of phenylephrine HCL is equivalent to 0.821 mg of phenylephrine free base).
³Acetone and purified water are removed during processing.

TABLE 13
Coated Phenylephrine Resinate Batch Formula

| | Weight | Weight % |
|---|---|---|
| Component | (kg/Batch) | (w/w) |
| Phenylephrine free base¹ | 7.41 | 24.7 |
| Sodium Polystyrene Sulfonate USP | 10.59 | 35.3 |
| (Amberlite ™ Ion-exchange Resin) | | |
| Cellulose Acetate NF | 9.00 | 30.0 |
| Hydroxypropyl Cellulose NF | 0.30 | 10.0 |
| Acetone NF² | — | — |
| Purified Water USP² | — | — |
| Total | 30.00 | 100 |

¹One mg of phenylephrine HCL is equivalent to 0.821 mg of phenylephrine free base.
²Acetone and purified water are removed during processing.

### Example 14

### Dissolution Analysis of Coated Phenylephrine Resin Particles

The coated phenylephrine resinate particles from Example 13 were tested for dissolution from 0 to 24 hours using the apparatus described in the United States Pharmacopeia General Chapter <711>, Dissolution, Apparatus II, rotating paddles, utilizing UV detection at 274 nm. The dissolution media was 750 mL of 0.1N HCL for the first hour and was 1000 mL 0.05M sodium phosphate buffer, pH 6.8, for the second to the 24^{th} hour. The temperature was 37° C. and rotation speed was 75 rpm. The dissolution showed that the percent released versus a standard prepared at 100% of the amount of phenylephrine in the formulation was less than or equal to 50% in 1 hour, greater than or equal to 30% in 3 hours and greater than or equal to 50% in 8 hours.

The following steps were followed for the Dissolution Method using USP Apparatus 2 (Paddles), 75 rpm:

1. Verify that the dissolution media temperature has reached the target value.

2. Add sample (onto the surface of the medium solution) to each vessel containing 750 mL of 0.1 N hydrochloric acid and start the dissolution test with the paddle speed at 75 rpm. After 1 hour of operation in 0.1 N hydrochloric acid, pull the 1 hour sample, and proceed immediately to the buffer stage by adding 250 mL of 0.20 M tribasic sodium phosphate. The pH of the media should be 6.8±0.05.

3. Pull 10 mL of dissolution sample solutions from each vessel after 1 hour, 2 Hours, 3 hours, 6 hours (optional), 8 hours, 12 hours and 24 hours. Filter the sample solutions through Varian Full Flow Filters (10 μm).

4. The amount of phenylephrine dissolved can be determined from UV absorbance in comparison with that of the standard solution at the wavelength of 274 nm. The amount of phenylephrine dissolved can also be determined using the phenylephrine assay method.

5. Correct the amount dissolved at 3, 6, and 8 hours by adding the amount pulled at the earlier time points. Use a dissolution program (or equivalent) or manually correct for intermediate sampling and removal of samples.

Table 14 is set forth below.

TABLE 4
Formulation Composition

| | Loading Drug/Resin Ratio | | | | | |
|---|---|---|---|---|---|---|
| | 1.33/1 | 1.33/1 | 1.33/1 | 1.33/1 | 0.59/1 | 1.60/1 |
| Coating level | 35.00% | 40.00% | 45.00% | 50.00% | 40.00% | 40.00% |
| Pheylephrine | 27.70% | 24.70% | 22.10% | 19.90% | 16.93% | 21.95% |
| (Base) | | | | | | |
| IRP-476 Resin | 37.30% | 35.30% | 32.90% | 30.10% | 43.07% | 38.05% |
| Cellulose | 26.25% | 30.00% | 33.75% | 37.50% | 30.00% | 30.00% |
| Acetate | | | | | | |
| Hydrocypropyl | 8.75% | 10.00% | 11.25% | 12.50% | 10.00% | 10.00% |
| Cellulose | | | | | | |

TABLE 15
Coating Solution Composition

| Material | % (w/w) |
|---|---|
| Cellulose Acetate NF | 4.50% |
| Hydroxypropyl Cellulose EF | 1.50% |
| Acetone NF | 84.60% |
| Purified Water USP | 9.40% |

Results

TABLE 16
Formulation for Tablet Containing 22.5 mg of
Drug from Coated Drug Resin (ER Portion) plus
7.5 mg of Phenylephrine HCL (IR Portion)

| | | | | | 7.5 mg |
|---|---|---|---|---|---|
| | | | | | of Drug |
| | 22.5 mg of Drug from ER | | | | from IR |
| | Coated Drug Loaded Resinate | | | | Phenyl- |
| | CA/HPC | Amount of | | | ephrine |
| | Coating | Coated | Amount of | Drug | HCL |
| | Level | Resinate | Resinate | Loaded | Amount |
| Tablet # | (%) | (mg) | (mg) | Resinate | (mg) |
| 1 | 40 | 74.8 | 26.4 | 3-Step | 7.5 |
| | | | | Loading | |
| | | | | Process | |
| Clinical* | 40 | 106.9 | 45.7 | 1-Step | 7.5 |
| | | | | Loading | |
| | | | | Process | |

*Coated drug resinate utilized for the clinical study

Discussion
After coating, the phenylephrine levels from the multiple-step loading remain higher than the single-step process at the same coating level (40%).
At a certain coating level, e.g. 40%, the higher phenylephrine loading level has a slightly faster release rate than the lower loading level.
Duplicated results were observed at the same phenylephrine loading and polymer coating level.
For Multiple-Step loading:
   The release rate is inversely proportional to the phenylephrine loading level, i.e., the higher the coating level (from 35% to 50%), the slower the release rate (83% to 42% at 2 hour time point).
   Release profile from a given single step loaded resinate can be matched with a corresponding multiple-step loaded resinate via an adjusted coating level.
The resin amount required in a single unit finished product can therefore be reduced from 45.7 to 26.4 mg and achieve the requirement to meet the published regulatory limit.

Conclusions

The results show that in the loading level ranges specified in the study, the phenylephrine HCl released from the coated polistirex particles was generally controlled by the coating level applied during the coating process while the loaded levels and numbers of steps applied in the drug loading process have no major impact. Minor adjustments of the coating levels and process parameters may be required, however, to achieve the same dissolution profile when switching from one loaded resinate to another.

With a similar release performance observed from this multiple-step, high loading phenylephrine coated resinate, a 12-hour sustained release of phenylephrine HCl formulation can therefore be achieved to comply with the excipient guideline on ion-exchange resin of 25 mg/day usage.

The foregoing examples are not intended to limit the scope of the present invention, which may be set out in the claims. In particular, various equivalents and substitutions will be recognized by those skilled in the art in view of the foregoing disclosure and these are contemplated to be within the scope of the invention.

### REFERENCES

Blackledge H M, O'Farrell J, Minton N A, et al. The effect of therapeutic doses of paracetamol on sulphur metabolism in man. Hum Exp Toxicol 1991 May; 10(3): 159-65.

Court M H, Duan S X, Von Moltke L L, et al. Interindividual variability in acetaminophen glucuronidation by human liver microsomes: Identification of relevant acetaminophen UDP-glucuronosyltransferase isoforms. J Pharmacol Exp Ther 2001; 299(3):998-1006.

Empey D W and Medder K T. Nasal Decongestants. Drugs 1981; 21:438-443.

Hengstmann J H, Goronzy J. Pharmacokinetics of 3H-phenylephrine in man. Eur J Clin Pharmacol 1982; 21:335-341.

Hoffman B B. Chapter 10: Catecholamines, Sympathomimetic Drugs, and Adrenergic Receptor Antagonists. In: Goodman & Gilman's The Pharmacologic Basis of Therapeutics-10th Ed. Hardman J G and Limbird L E, eds. McGraw-Hill, Medical Publishing Division, USA, 2001.

Ibrahim K E, Midgley J M, Crowley I R, and Willaims C M. The mammalian metabolism of R-(-)-m-synephrine. J Pharm Pharmacol. 1983; 35:144-147.

Johnson D A, Hricik J G. The pharmacology of a-adrenergic decongestants. Pharmacother 1993; 13:110S-115S.

Koch-Weser J. Medical Intelligence: Drug Therapy. N Engl J Med 1976 Dec. 2; 295(23):1297-1300.

Manyike P T, Kharasch E D, Kalhorn T F, et al. Contribution of CYP2E1 and CYP3A to acetaminophen reactive metabolite formation. Clin Pharmacol Ther 2000 March; 67(3):275-282.

Miners J O, Atwood J, Birkett D J. Influence of sex and oral contraceptive steroids on paracetamol metabolism. Br J Clin Pharmacol 1983; 16:503-509.

Miners J O, Osborne N J, Tonkin A L, et al. Perturbation of paracetamol urinary metabolic ratios by urine flow rate. Br J Clin Pharmacol 1992; 34:359-362.

Mitchell J R, Thorgeirsson S S, Potter W Z, et al. Acetaminophen-induced injury: Protective role of glutathione in man and rationale for therapy. Clin Pharmacol Ther 1974; 16:676-684.

Slattery J T, McRorie T I, Reynolds R, et al. Lack of effect of cimetidine on acetaminophen disposition in humans. Clin Pharmacol Ther 1989 November; 46(5):591-597.

Suzuki O. Matsumoto T. Oya M, Katsumata Y. Oxidation of synephrine by type A and type B monoamine oxidase. Experientia 1979; 35:1283-1284.

1. A drug-resin complex comprising phenylephrine and a cation polystyrene sulfonate, wherein said cation polystyrene sulfonate comprises particle sizes of about 74 μm to about 177 μm prior to being combined with the phenylephrine, and wherein said drug resin complex comprises phenylephrine:resin in a ratio of greater than about 1:1.

2. The drug-resin complex of claim 1, wherein the cation is selected from the group consisting of sodium, copper, zinc, iron, calcium, strontium, magnesium and lithium.

3. The drug-resin complex of claim 2, wherein the cation is sodium.

4. An extended release particle, wherein said extended release particle comprises the drug-resin complex of claim 3 coated with a coating.

5. The extended release particle of claim 4, wherein the coating comprises a cellulose material.

6. The extended release particle of claim 5, wherein the cellulose material is selected from the group consisting of cellulose acetate and hydroxypropylcellulose.

7. A pharmaceutical formulation comprising the extended release particle of claim 6.

8. The pharmaceutical formulation of claim 7, further comprising an immediate release form of phenylephrine.

9. A method of forming a coated drug-resin complex, comprising coating the drug-resin complex of claim 1.

10. The drug-resin complex of claim 1, wherein at least about 50% of the particles have particle sizes of about 74 μm to about 177 μm.

11. The drug-resin complex of claim 10, wherein at least about 80% of the particles have a particle sizes of about 74 μm to about 177 μm.

12. The drug-resin complex of claim 11, wherein at least about 90% of the particles have a particle sizes of about 74 μm to about 177 μm.

13. The drug-resin complex of claim 1, wherein less than 15% of the particles have a particle size less than about 44 μm.

14. The drug resin complex of claim 1, wherein said drug resin complex comprises phenylephrine:resin in a ratio of about 1.25:1.

15. The drug resin complex of claim 1, wherein said drug resin complex comprises phenylephrine:resin in a ratio of about 1.33:1.

16. The drug resin complex of claim 1, wherein said drug resin complex comprises phenylephrine:resin in a ratio of about 1.4:1.

17. The drug resin complex of claim 1, wherein said drug resin complex comprises phenylephrine:resin in a ratio of about 1.5:1

18. The drug resin complex of claim 1, wherein said drug resin complex comprises phenylephrine:resin in a ratio of about 1.6:1

19. A process for preparing a drug-resin complex, comprising:
Step A:
mixing purified water and resin in a container to form a first mixture;
filtering the first mixture to form a wet cake containing resin;
rinsing the wet cake containing resin with purified water;
filtering the rinsed wet cake containing resin;
Step B:
mixing purified drug and water in a container to form a drug solution;
Step C:
mixing the filtered wet cake containing resin and a portion of the drug solution in a container to form a second mixture;
filtering the second mixture to form a first loaded resinate;
Step D:
mixing the first loaded resinate and a portion of the drug solution in a container to form a third mixture;
filtering the third mixture to form a second loaded resinate;
Step E:
optionally, repeating step D multiple times; and
Step F:
drying the loaded resinate to form the drug-resin complex.

20. The process of claim 19, wherein the drug is phenylephrine.

21. The process of claim 20, wherein the resin is a cation polystyrene sulfonate.

22. The process of claim 21, wherein the cation polystyrene sulfonate comprises particle sizes of about 74 μm to about 177 μm prior to being combined with the phenylephrine.

23. The process of claim 22, wherein the drug resin complex comprises phenylephrine:resin in a ratio of greater than about 1:1

24. The process of claim 23, wherein the drug resin complex comprises phenylephrine:resin in a ratio of about 1.4:1
